(19) United States
(12) Patent Application Publication
(10) Pub. No.: US 2026/0269087 A1
(43) Pub. Date: Sep. 10, 2026

(54) 24/7 MEDICAL TRIAGE AND COORDINATION SYSTEM FOR PHYSICIAN PRACTICES

(71) Applicants: Nicholas Tarazi, Clovis, CA (US); Mona Navarro, Clovis, CA (US)

(72) Inventors: Nicholas Tarazi, Clovis, CA (US); Mona Navarro, Clovis, CA (US)

(21) Appl. No.: 19/072,439

(22) Filed: Mar. 6, 2025

(51) Int. Cl. G16H 80/00; G06Q 40/08; G16H 10/60; G16H 40/20; G16H 50/20

(52) U.S. Cl. CPC G16H 80/00; G06Q 40/08; G16H 10/60; G16H 40/20; G16H 50/20

(57) ABSTRACT

A software-driven system for continuous triage and medical coordination integrates real-time AI-powered patient inquiry categorization, EMR access, and direct physician oversight. The system includes a computing device operably connected to a network, a patient interface for receiving calls and messages, and an AI-driven triage module for determining urgency and routing cases. Physicians maintain full control over patient care, billing, and liability while ensuring seamless communication with patients. The system enables multi-payer reimbursement, direct employer contracts, and Medicare integration, bypassing insurer restrictions. A legal compliance module records interactions for liability protection, while an encrypted communications module ensures HIPAA compliance. By maintaining the perception of direct patient-physician engagement, the system operates as a virtual extension of a physician's practice and the physician's license, ensuring 24/7 accessibility without requiring insurer pre-approvals or disrupting continuity of care.

### TECHNICAL FIELD

The embodiments generally relate to the technical field of AI-driven medical triage and coordination within physician practices. More specifically, the disclosed system integrates real-time patient inquiry management, automated triage decision-making, and seamless interoperability with electronic medical records (EMR) to enhance continuous patient care and physician oversight.

### BACKGROUND

Conventional systems for managing doctor's office patient inquiries and medical coordination is essential for maintaining high-quality healthcare, particularly in physician practices where timely responses can significantly impact patient outcomes. Traditional systems for handling patient calls and triage rely heavily on manual processes, such as answering services or call-back models, which can lead to delays in care, miscommunication, and increased administrative burdens on healthcare providers. These conventional approaches often fail to provide real-time decision-making and lack integration with EMR, resulting in inefficiencies and gaps in patient management.

While telemedicine solutions have improved accessibility to healthcare, many existing platforms operate on a limited callback basis or require insurer pre-approvals, restricting immediate patient access to medical assistance. Additionally, these solutions often function as standalone services rather than fully integrating with a physician's practice, leading to fragmented patient care and reduced continuity in treatment plans. The reliance on external systems also limits physicians' control over billing, reimbursement, and liability protections.

Furthermore, emergency medical services (EMS) and traditional 911 dispatch systems are frequently used for non-emergent cases due to a lack of alternative triage pathways. Studies show that a significant percentage of ambulance calls, and emergency room visits are preventable, placing unnecessary strain on healthcare resources. Without an integrated, physician-led triage system, patients lack a structured approach to determining whether their medical concerns require emergency care, an office visit, or remote management. The need exists for a comprehensive, AI-driven triage and medical coordination system that seamlessly extends a physician's practice, offering continuous, real-time patient management while maintaining control over care delivery, billing, and liability.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

A software-driven system and method for AI-powered triage and medical coordination is disclosed that enables continuous, real-time patient inquiry management within a physician's practice. The system integrates artificial intelligence (AI)-driven triage, EMR interoperability, and physician oversight to facilitate immediate patient response without requiring insurer pre-approvals.

The system includes a computing device that receives patient inquiries through multiple communication channels, including phone, SMS, and digital applications such as Internet of Things (IOT) devices, such as smartphones, smartwatches, smart rings, etc., and medical-related IoT devices, such as smart blood pressure monitors, glucometers, ECG monitors, defibrillators, pulse oximeters, etc. An AI-driven triage module analyzes and categorizes patient concerns based on urgency and medical context, determining whether self-care, physician consultation, or escalation is required. The system updates EMR records in real time and allows physicians to set triage protocols, view patient escalations, and intervene when necessary.

A legal compliance module records all patient interactions to provide liability protection, ensuring a defensible medical record for physicians. Additionally, a billing and insurance processing module supports direct physician-led reimbursement models, including Medicare, private insurance, and employer healthcare contracts. The system maintains the perception of direct patient-physician engagement, functioning as an always-available extension of a physician's office while reducing unnecessary emergency room visits and administrative burdens.

In some embodiments, the system integrates medical adherence and remote patient monitoring (RPM) by leveraging AI-driven analytics, IoT medical devices, and real-time data synchronization with electronic medical records (EMR). The system ensures medication compliance by tracking patient adherence, detecting anomalies, and notifying healthcare providers when intervention is required.

By leveraging AI-driven decision-making, real-time communication, and physician-led billing structures, the system optimizes patient care accessibility, streamlines triage processes, and enhances practice revenue generation, ensuring seamless healthcare coordination beyond traditional office hours.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram, according to some embodiments;

FIG. 2 illustrates an application program and modules in communication with the computing system, according to some embodiments;

FIG. 3 illustrates a block diagram of a 24/7 medical triage and coordination system for physician practices, according to some embodiments;

FIG. 4 illustrates a block diagram of a 24/7 medical triage and coordination system for physician practices including an AI-driven patient triage and routing process, according to some embodiments; and

FIG. 5 illustrates a flowchart diagram of a 24/7 medical triage and coordination system for physician practices, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The disclosed system is a software-driven medical coordination platform designed to provide continuous, AI-enhanced triage and patient management while maintaining real-time physician oversight. The system operates on a network-connected computing device and incorporates multiple modules to perform various medical triage, billing, and communication functions.

A patient interface serves as the entry point for patient interactions and is implemented via a cloud-based VoIP system, SMS gateway, mobile application, and web-based portal. This interface is managed by a patient communications module, which utilizes speech recognition software, natural language processing (NLP), and interactive voice response (IVR) technology to interpret patient inquiries and route them to the appropriate next step. A backend call-routing server ensures that urgent inquiries are prioritized.

An AI-driven triage module employs machine learning models trained on extensive medical datasets to classify patient inquiries based on urgency and medical relevance. This module operates through a neural network-based decision engine that evaluates input data, including reported symptoms, patient history, and risk factors. It assigns each case a triage score and determines whether immediate intervention is required. Bayesian inference and decision trees aid in refining diagnoses. The triage module also interfaces with a database of standardized medical protocols to recommend follow-up actions for non-emergent cases.

An EMR integration module ensures real-time interoperability between the triage system and third-party EMR platforms, such as Epic and Cerner. This module is implemented using standardized HL7 and FHIR API protocols, enabling seamless data exchange. The integration module retrieves relevant patient histories, medication lists, and prior visit records, ensuring that physicians and triage personnel have access to comprehensive patient data. Additionally, an automated documentation system updates the EMR with triage notes and recommended actions.

A physician control module provides a graphical user interface (GUI) that allows physicians to oversee triage decisions, access patient records, and intervene in high-priority cases. The module is built on a secure web-based dashboard with real-time notifications, enabling physicians to review AI-generated recommendations and override decisions when necessary. The control module also allows configuration of triage rules and escalation thresholds to tailor the system's operation to each physician's practice.

A billing and insurance processing module automates financial transactions related to triage and patient care services. This module utilizes an integrated claims management system that supports multi-payer billing, including private insurers, Medicare, and direct employer contracts. The module applies AI-based medical coding to assign appropriate CPT and ICD-10 codes based on triage outcomes, ensuring proper reimbursement. It connects to external insurance verification systems via secure APIs to process claims efficiently.

A legal compliance and liability protection module ensures that all patient interactions, including triage calls, physician consultations, and treatment recommendations, are securely recorded and stored for auditing and liability protection. This module uses AES-256 encryption to safeguard sensitive data and employs audit logging to create an immutable record of interactions. In embodiments, the legal compliance and liability protection module is configured to integrate blockchain-based and off-chain audit logging to provide an immutable record of interactions. Blockchain integration ensures that critical compliance events, such as contract approvals, policy changes, or user agreements, are securely logged with cryptographic hashing, making them tamper-proof and verifiable. Additionally, off-chain logging may be used to store identical information to that stored on the blockchain, or larger, less critical compliance records efficiently, reducing blockchain storage overhead while maintaining integrity through cryptographic linking. By combining AES-256 encryption with blockchain or off-chain auditing, the system enhances security, prevents data breaches, and ensures a transparent and legally defensible record of compliance-related activities. The legal compliance and liability protection module may include a user access control system that restricts data access to authorized personnel only, ensuring compliance with HIPAA and GDPR regulations.

A communication module is designed to facilitate seamless, real-time interaction between patients and healthcare providers while maintaining the perception of direct engagement with the physician's office. This module employs voice synthesis technology to generate automated responses that mimic a physician's voice, ensuring continuity in patient communication. Masked call routing technology ensures that outbound patient calls appear as originating from the physician's office, preserving trust and familiarity. In embodiments, the communication module is configured to facilitate communication between devices via over wired connections (e.g., Ethernet, USB, serial communication, fiber optics, etc.) or wireless protocols (e.g., Wi-Fi, Bluetooth, cellular networks, satellite, RFID/NFC, etc.).

A real-time alerting mechanism built into the system notifies designated healthcare providers of high-priority cases requiring immediate intervention. The alert system is integrated with push notifications, SMS alerts, and direct EMR notifications. The alerting algorithm uses predefined rules and adaptive learning models to refine escalation protocols based on past interactions and patient-specific risk factors. In embodiments, the real-time alerting mechanism is configured to communicate, via the communication module, with IoT devices, such as, but not limited to smartphones, smartwatches, smart rings, etc., and medical-related IoT devices, such as smart blood pressure monitors, glucometers, ECG monitors, defibrillators, pulse oximeters, sleep trackers, fall detection monitors, smart insulin pens, etc., in order to communicate or receive push notifications, SMS alerts, and direct EMR notifications. The system may be configured for medical adherence functionality connects with patients via a mobile application that syncs with IoT devices such as smart pill dispensers, wearables, and Bluetooth-enabled biometric sensors, such as blood pressure monitors, glucose meters, and pulse oximeters. When a patient takes or misses a scheduled dose, the system logs the event, cross-referencing it with prescribed treatment plans stored in the EMR. AI algorithms analyze adherence patterns and predict potential non-compliance risks, sending automated reminders to patients or escalating alerts to caregivers and physicians if deviations persist.

The system architecture is designed for scalability and adaptability across diverse patient populations. It includes multilingual support, accessibility features for individuals with disabilities, and adaptive AI models that update based on evolving medical guidelines. The system leverages cloud-based infrastructure with distributed computing capabilities to handle high patient interaction volumes.

By integrating AI-driven triage, EMR interoperability, real-time physician oversight, and automated billing, the disclosed system transforms traditional medical practice into a fully operational 24/7 care coordination platform. It optimizes patient care accessibility, reduces administrative burdens, minimizes unnecessary emergency visits, and enhances physician revenue through efficient billing and reimbursement processes.

Various implementations of the invention involve the technical field of AI-driven medical triage and coordination including an artificial intelligence (AI)-driven triage module configured to categorize patient inquiries, determine urgency, and route cases to appropriate medical personnel; an electronic medical record (EMR) integration module that allows real-time access, retrieval, and updating of patient medical histories by authorized personnel; a billing and insurance processing module configured to support multi-payer reimbursement, direct employer contracts, and Medicare integration; a legal compliance and liability protection module that records patient interactions and maintains auditable logs; and a communications module enabling seamless, real-time patient-physician interactions while preserving the perception of direct engagement with the physician's office and are therefore necessarily rooted in computer technology. For example, the aforementioned steps are inherently computer-based and cannot be performed in the human mind. The present invention amounts to more than merely implementing the generic computer as a tool to gather, analyze, and output data because the system introduces a fundamentally improved medical coordination platform that integrates AI-driven triage, real-time physician oversight, and automated medical decision-making. Unlike traditional systems that passively store and retrieve information, this system actively processes patient inquiries using a neural network-based decision engine, applies Bayesian inference to refine triage outcomes, and directly integrates with EMR platforms in real-time. Additionally, the steps of the present invention would be impossible to accomplish on pen and paper due to the volume of data being communicated and received over a network in real-time. In particular, the speed at which the steps of the present invention occur to effectuate the disclosed method, system, or product would involve large-scale, continuous wireless communication of such data. That is, the steps of the present method, system, or product are impossible to accomplish on pen and paper, cannot be accomplished as a method of organizing human activity, and amount to significantly more than merely gathering, analyzing, and outputting data.

Implementations of the present invention include implementing (executing, running, or deploying) one or more artificial intelligence models on a computing device wherein the computing device executes the artificial intelligence model's algorithms and mathematical functions on computer hardware using machine learning libraries. The computing device implements the artificial intelligence model when it performs tasks like training, making predictions, applying the model to data, decision-making, classification, or generating outputs based on inputs. In particular, the speed at which an artificial intelligence model analyzes and transforms data to effectuate the disclosed method, system, or product would involve large-scale, continuous transformation of such data. As such, the present invention would be impossible to accomplish on pen and paper or in the human mind due to the volume of data being analyzed and transformed by the artificial intelligence model.

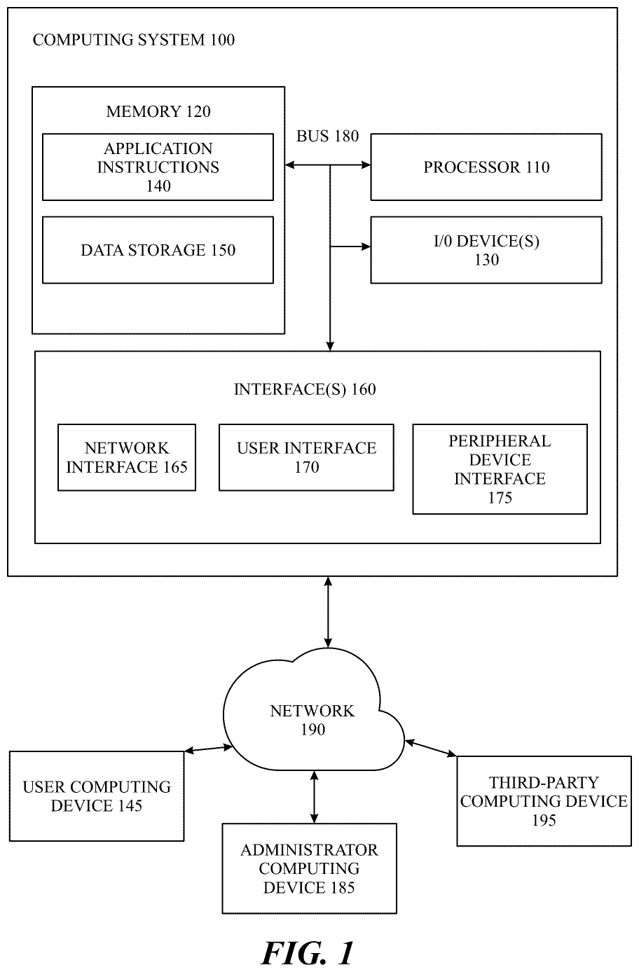

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computer system 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 140, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C #, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized by a user to interact with the various functionalities of the system. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

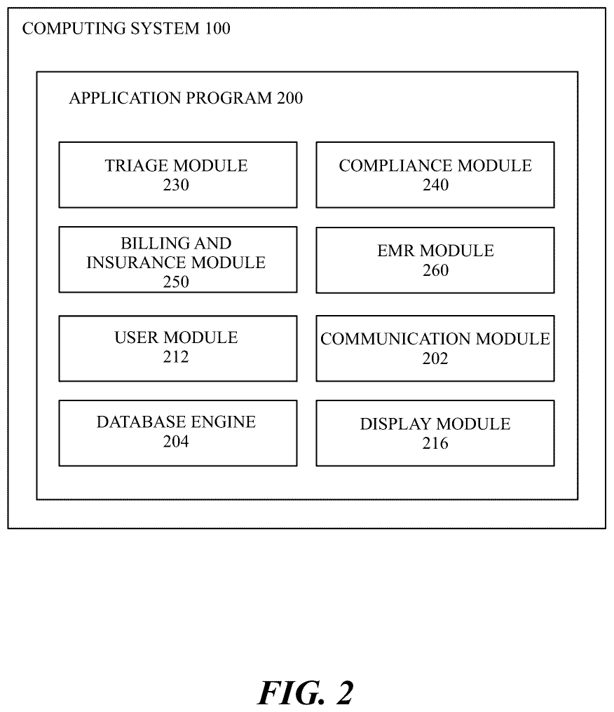

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a triage module 230, a compliance module 240, a billing and insurance module 250, an EMR module 260, a communication module 202, a database engine 204, a user module 212, and a display module 216.

In some embodiments, the compliance module 240 is configured to ensure secure data handling, regulatory adherence, and liability protection by encrypting, recording, and auditing all patient interactions. The compliance module 240 encrypts data using AES-256 encryption and employs secure API gateways for communication between system components. Multi-factor authentication (MFA) controls access to sensitive patient records, ensuring only authorized personnel can review or modify data.

To maintain compliance with HIPAA, GDPR, and HITECH regulations, the compliance module 240 anonymizes patient data before training AI models, such as AI modules within the triage module. The compliance module 240 logs all triage decisions, physician interventions, and patient communications using a blockchain-based and off-chain audit system(s), creating an immutable record of events. This log allows physicians and administrators to track decision-making processes and provides a legal safeguard in case of disputes.

The compliance module 240 may be configured to record all voice calls, SMS messages, and digital interactions, storing them in a secure, cloud-based repository with automatic retention policies. The compliance module 240 may be configured to record data access and modification in real time, triggering alerts if unauthorized changes occur. Additionally, the compliance module 240 integrates with legal compliance tools to generate automated reports for audits and insurer reviews.

In some embodiments, the billing and insurance module 250 is configured to automate claims processing, payment verification, and reimbursement tracking, ensuring seamless financial transactions for medical services. The billing and insurance module 250 integrates with private insurers, Medicare, and employer-sponsored healthcare plans via secure API connections. Upon triage completion, the module assigns CPT and ICD-10 codes via AI modules within the triage module, which analyzes patient interactions and EMR data to ensure accurate billing.

In some embodiments, the EMR module 260 is configured to integrate information between the triage module 230 and EMR platforms, ensuring real-time data access, retrieval, and updates. The EMR module 260 connects to third-party EMR systems, such as Epic and Cerner, using standardized HL7 and FHIR API protocols. The EMR module 260 retrieves patient histories, including prior diagnoses, medications, and allergies, and encrypts all transmitted data using AES-256 encryption to maintain HIPAA compliance.

Upon receiving a patient inquiry, the EMR module 260 queries the EMR to provide the triage module 230 with relevant medical context. The EMR module 260 updates patient records automatically based on triage module 230 outcomes, physician interventions, and prescribed treatments. The EMR module 260 employs caching mechanisms to optimize data retrieval speeds, reducing latency for high-volume interactions.

The EMR module 260 logs all EMR modifications using a blockchain or off-chain-based audit trail, ensuring transparency and accountability in medical decision-making. Physicians can review triage decisions, override AI recommendations, and add clinical notes through a secure web-based dashboard. The EMR module 260 also synchronizes follow-up appointments, prescription requests, and referrals with the EMR, ensuring continuity of care.

In some embodiments, the triage module 230 is configured to optionally handle patient calls by assessing patient inquiries, determining urgency, and routing cases in real time. The triage module 230 integrates NLP, machine learning, and rule-based logic while connecting with the EMR module 260 to ensure informed decision-making and regulatory compliance.

Patient inquiries may arrive via VoIP, SMS, mobile apps, or web portals and are processed through an IVR system with speech-to-text conversion. The system may retrieve patient history via HL7 and FHIR protocols, encrypting data exchanges through a secure API gateway. NLP algorithms analyze symptoms using Named Entity Recognition (NER) and sentiment analysis to assess urgency.

The triage module 230 may include an AI decision engine that employs transformer-based NLP models, decision trees, and Bayesian inference to classify conditions based on patient information, such as spoken voice-input. A rule-based expert system applies clinical guidelines, and an urgency scoring algorithm ranks cases, such as from 1 to 100. Cases may be correlated to self-care guidance, routed to a physician, or may trigger EMS dispatch depending on the calculated score. In some embodiments, physicians or system administrators oversee AI recommendations through a dashboard, allowing real-time review and intervention. The triage module 230 may encrypt data using AES-256, requires MFA for access, and records interactions via blockchain or off-chain processes for compliance with HIPAA, GDPR, and HITECH.

In some embodiments, the communication module 202 is configured for receiving, processing, and transmitting patient or user calls or user commands and/or one or more data streams. The communication module is configured to intercept patient calls to a doctor's office after-hours and redirect the call to a third-party call service such that the patient's call may be addressed by a third-party servicer, and medical service may be provided to a patient. The communication module 202 is configured to use call forwarding or call routing via carrier based call forwarding, PBX, VoIP systems, SIP trunking, or other system. In this way, a patient may receive medical care from their doctor's office after traditional working hours. In such embodiments, the communication module 202 performs communication functions between various devices, including the user computing device 145 of FIG. 1, the administrator computing device 185 of FIG. 1, and a third-party computing device 195 of FIG. 1. In some embodiments, the communication module 202 is configured to allow one or more users of the system, including a third-party, to communicate with one another. In some embodiments, the communications module 202 is configured to maintain one or more communication sessions with one or more servers, the administrative computing device 185 of FIG. 1, and/or one or more third-party computing device(s) 195 of FIG. 1. In some embodiments, the communication module 202 may allow users and administrators to communicate with one another. In embodiments, the communication module 202 is configured to provide medical adherence functionality operates through a mobile application that establishes encrypted Bluetooth Low Energy (BLE) connections with smart pill dispensers, wearable devices, and biometric sensors such as blood pressure monitors, glucose meters, and pulse oximeters. The application communicates with these devices using standardized protocols such as Bluetooth GATT (Generic Attribute Profile) to capture real-time adherence data. Each medication event—whether a dose is taken, skipped, or delayed—is time-stamped and logged in a secure database. The system cross-references these records with prescribed treatment plans retrieved from the EMR via HL7 or FHIR API protocols, ensuring adherence tracking aligns with physician-prescribed regimens.

The AI decision engine processes adherence data using machine learning models trained on historical patient behavior. The system detects deviations from expected adherence patterns, applies predictive modeling to assess the risk of medication non-compliance, and generates intervention strategies. If the system predicts potential non-adherence, it triggers automated multi-channel reminders via push notifications, SMS, or voice calls. For high-risk cases, the system escalates alerts to caregivers and healthcare providers, integrating with remote monitoring dashboards to provide real-time adherence insights.

For RPM, the system continuously collects and transmits physiological data from Bluetooth-connected devices to a cloud-based repository. The AI decision engine may process this data, identifying trends and potential health deteriorations. If abnormal readings are detected—such as irregular heart rate, dangerously high blood glucose levels, or sudden weight fluctuations —the system may alert healthcare providers, enabling proactive intervention.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations.

The user module 212 may store user preferences including the user account information, historical usage data, user personal information, and the like. The user module 212 may facilitate the creation of user's profiles for users, administrators, and others.

In some embodiments, the display module 216 is configured to display one or more graphic user interfaces, including, e.g., one or more user interfaces. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored. The display module 216 displays information, notifications, and alerts to the user device which can be viewed and acknowledged by the user.

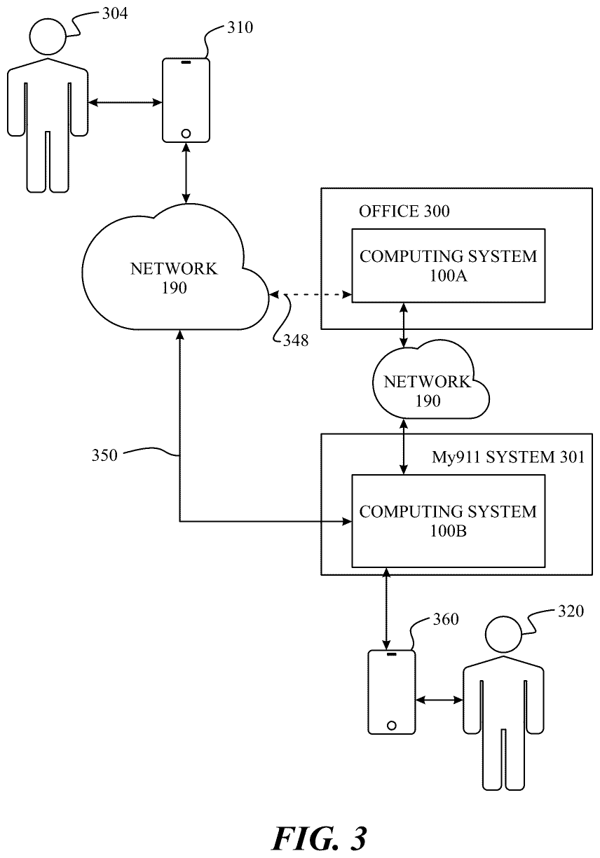

FIG. 3 illustrates a block diagram of a 24/7 medical triage and coordination system “My911” system 301 for physician practices implemented by a doctor's office 300 in communication with one another via computing systems 100A, 100B over network 190. A patient 304 of the office 300 may call 348 the office 300 via their phone 310 over network 190. The system may be configured to monitor calls 348 after office 300 hours and redirect 350 calls to the system 301 including system phone 360 to be handled by an operator 320 or the triage module 230 of the computer system 100B, as described with respect to FIG. 2. In this way, the system 301 handles patients calls 348 for the doctor's office 300 during non-working hours.

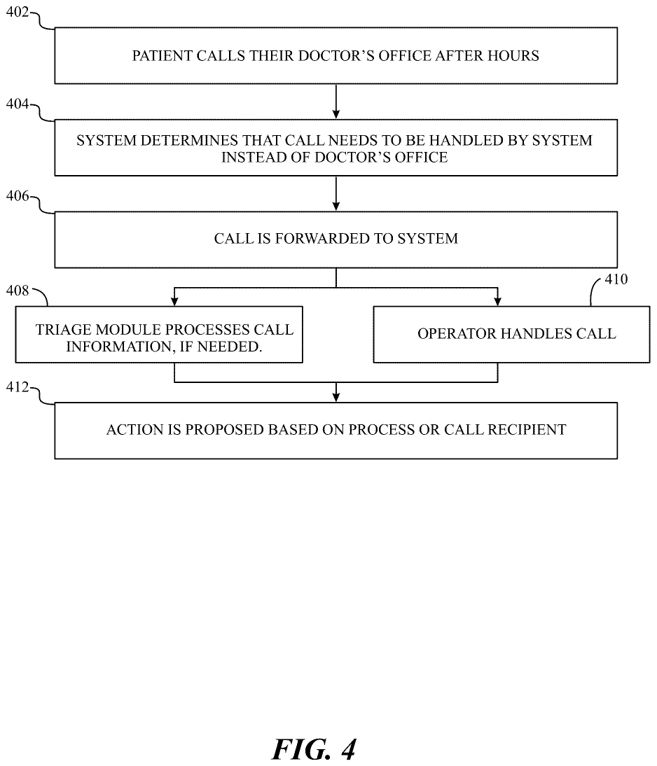

FIG. 4 illustrates a block diagram of a 24/7 medical triage and coordination system for physician practices including an AI-driven patient triage and routing process. In step 402, a patient call's their traditional, “brick-and-mortar” doctor's office with, for example, a question regarding a medical condition, injury, or an emergency. In step 404, the system identifies the call as being after hours. For example, the patient's call occurs at 1:00 AM and is a non-life-threatening emergency. However, the doctor's office is not staffed at 1:00 AM. In step 406, and in response to the system identifying the call as being after hours, the call is forwarded to the system and the system automatically routes the call to an operator and, in step 410, the operator handles the call by providing medical advice including updating the patient's medical record on behalf of the doctor's office. The operator may also, in step 412, propose remedial action to the patient, or in some circumstances, dispatch emergency services such as ambulance services. In some embodiments, in step 408, the system is configured to route calls to the triage module for AI-based processing in place of an operator handling the call. The triage module may also, in step 412, propose remedial action to the patient, or in some circumstances, dispatch emergency services such as ambulance services.

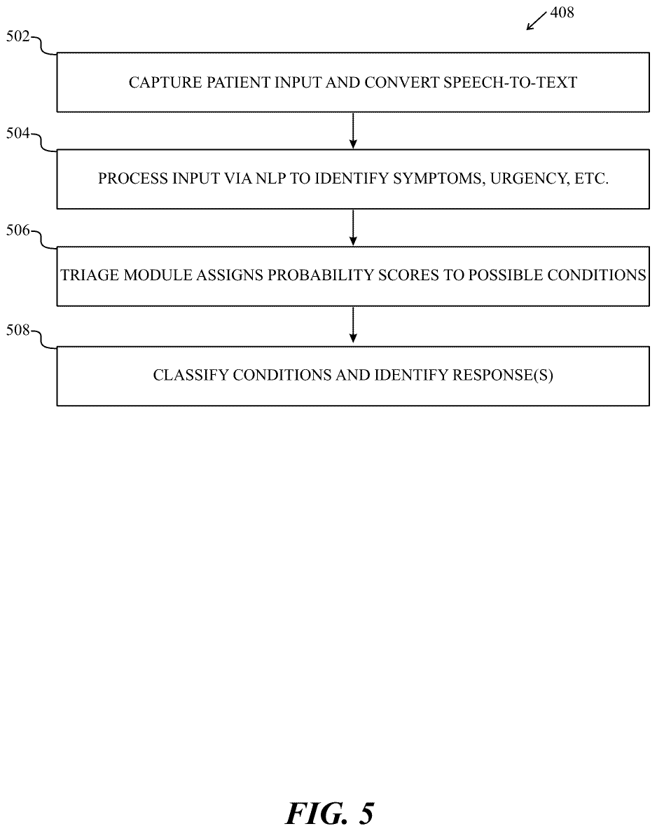

FIG. 5 illustrates a flowchart diagram of a 24/7 medical triage and coordination system for physician practices, and more specifically, step 408 of FIG. 4. When a patient calls their traditional doctor's office with, for example, a question regarding a medical condition, injury, or an emergency, the triage module may, in step 502, capture patient audio input and convert audio data to text via speech-to-text conversion. The system may, in step 504, use NLP to process patient text data to identify patient symptoms, urgency, emergency situations, etc. In step 506, the triage module may employ an AI decision engine that employs transformer-based NLP models, decision trees, or Bayesian inference to classify conditions based on patient information, such as spoken voice-input processed to text. A rule-based expert system applies clinical guidelines, and an urgency scoring algorithm ranks cases, such as from 1 to 100. In step 508, cases may be correlated to self-care guidance, routed to a physician, or may trigger EMS dispatch depending on the calculated score.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrases “computing device” or “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

1-20. (canceled)

21. A software-driven system for integrating 24/7 triage and medical coordination into a physician's practice as a true virtual extension of the physician's full state-licensed medical practice, the system comprising:
(1) a computing device operably connected to a network;
(2) a patient interface configured to receive incoming patient communications via at least one of a voice-over-IP (VoIP) system, SMS gateway, mobile application, IoT devices, or web-based portal, and route them through an automated triage system, wherein the system is audio-first such that initial patient contact is established via audio and safety assessment—including ambulance dispatch confirmation when clinically indicated—is completed prior to any further escalation;
(3) an ambient listening module configured to capture, during a live triage interaction, all verbal disclosures and acoustic features of the patient across a frequency spectrum broader than the range of unaided human hearing—comprising acoustic signals in the infrasound range below 20 Hz, the full audible range from 20 Hz to 20,000 Hz, and optionally extended frequencies above 20,000 Hz—wherein the captured acoustic features include lung sounds, breathing rate, voice tensions, and coughing patterns at frequencies that may be inaudible to or undetected by the human triage person, the module further configured to: (i) classify the captured acoustic features against a predefined clinical acoustic taxonomy informed by established clinical literature on lung sounds; (ii) identify verbal disclosures made by the patient that were not incorporated into the triage person's documented assessment and flag those unaddressed disclosures for clinical review; and (iii) generate real-time prompts and suggested phrasing delivered to the triage person during the live interaction without interrupting conversation flow, wherein the module operates as a passive decision-support layer assisting the human triage person rather than replacing human clinical judgment;
(4) an artificial intelligence (AI)-driven triage module configured to authenticate patient identity and insurance eligibility in real time, categorize patient inquiries, determine urgency via an urgency scoring algorithm, and route cases to appropriate medical personnel, wherein the AI-driven triage module is configurable by the physician via a secure GUI dashboard to align with practice-specific patient management protocols;
(5) a video escalation and real-time vital signs capture module configured to, upon confirmation that emergency dispatch is not required, escalate the interaction from audio to an on-demand WebRTC video session and capture real-time vital signs data from at least one of a camera-based contactless source or a device-integrated wearable or IoT sensor source, wherein all captured vital signs data is uploaded in real time to the patient's persistent data folder for concurrent AI and human review prior to integration into the electronic medical record;
(6) a patient data interoperability module configured to structure patient information as comprehensive persistent patient data folders—as distinguished from traditional field-based database records—containing full patient history, triage outcomes, vital signs data, and interaction records, and further configured to enable portability of the patient data folder via QR codes and shareable secure links for transmission to other licensed medical professionals;
(7) an empathy measurement and support module configured to classify triage staff speech and behavior in real time against a predefined structured taxonomy of discrete emotional states organized by emotional category and intensity gradient, apply the RADAR listening formula (a structured four-component empathic response comprising a tentative reflective opening, a classified feeling word, a contextualizing connector, and a reflected summary of the patient's expressed concern) to generate scored empathy response patterns, produce a quantified Empathy Score per interaction and per staff member accumulated over time, flag Empathy Score degradation below a configurable threshold to trigger a supervisory alert or wellness intervention, detect discrepancies between the triage person's pain assessment and the patient's AI-detected severity to provide real-time calibration feedback that trains the triage person to distinguish hearing from listening and to align their emotional response with objective data—thereby reducing emotional mirroring bias and improving triage accuracy and staff resilience whether the triage person is a volunteer, a retired paramedic, or paid clinical staff—and incorporate built-in trauma-informed safeguards to ensure that neither the human triage person nor the AI system becomes dominant, manipulative, or dismissive when interacting with vulnerable patients experiencing pain or emotional distress, while respecting the patient's voice, choice, and comfort level with AI involvement; and generate, track, and act upon Key Performance Indicators (KPIs) comprising at least empathy identification accuracy rate, emotional validation rate per interaction, staff resilience score over time, care continuity metric, and patient User Experience (UX) score;
(8) an electronic medical record (EMR) integration module configured to allow real-time access, retrieval, and updating of patient medical histories by authorized personnel, automatically append AI-generated triage notes and captured vital signs data into the EMR as auditable structured data via HL7/FHIR interoperability standards, and map triage-generated data to specific FHIR R4 resource types in a non-conventional manner, comprising at least: (i) RiskAssessment resources populated with urgency scores using prediction. outcome and probability fields; (ii) Observation resources for vital signs and Empathy Scores using category value survey; (iii) Encounter resources for escalation events with encounter. class and encounter. priority fields populated directly from the urgency scoring algorithm output; and (iv) DocumentReference resources for AI-generated triage notes;
(9) a physician control module that enables direct physician oversight of triage decisions, configuration of practice-specific protocols, real-time visibility into triage activity and outcomes, and physician override authority across all system functions;
(10) a billing and insurance processing module configured to apply AI-based medical coding to assign CPT and ICD-10 codes based on triage outcomes produced by the AI-driven triage module, cross-validate those assigned codes against payer-specific claim rules prior to submission, route validated billing data to the applicable payer channel based on the patient's identified coverage type—including private insurance carriers, Medicare, Medicaid, Veterans Administration programs, Workers' Compensation carriers, and direct employer contracts—via secure payer API connections, and present assigned codes to the physician for review and override prior to final claim submission;
(11) a legal compliance and liability protection module that encrypts patient interactions using AES-256 encryption and records every triage interaction as an immutable entry on a blockchain-backed ledger with cryptographic hashing, providing verifiable audit trails of all triage decisions, Empathy Scores, vital signs captures, and escalation events; and
(12) a communications module enabling real-time patient-physician interactions while maintaining the patient's perception of direct engagement with the physician's office; wherein the system operates as an always-available virtual extension of the physician's full state-licensed medical practice across the entire state or states in which the physician holds an active medical license, without geographic limitation to specific zip codes or localities, training, teaching, implementing, and measuring protocol-compliant, empathetic 24/7 triage and coordination at scale under the core competencies of Advice, Refer, and Prescribe, with immutable audit logs stored on a blockchain-backed ledger, and wherein the system generates machine-readable, quantified Empathy Scores and triage outcome records to enable continuous, measurable quality improvement across staff empathy performance and clinical triage accuracy.

22. The system of claim 21, wherein the ambient listening module is further configured to classify captured lung sounds against a multi-dimensional acoustic taxonomy comprising at least sound type, echo character, depth indicator, and associated organ system correlation—informed by foundational clinical research on multiple distinct lung sounds and their links to chronic and acute conditions including heart disease—and to update the patient's persistent data folder with the classified respiratory indicators in real time during the triage interaction.

23. The system of claim 21, wherein the video escalation and real-time vital signs capture module operates in a proactive remote patient monitoring (RPM) or personal emergency response system (PERS) mode for preventative monitoring in addition to reactive triage, and wherein the system initiates proactive outreach comprising targeted clinical questions when patient data patterns indicate elevated risk.

24. The system of claim 21, wherein the patient data interoperability module allows the patient to control the sharing of their persistent data folder with licensed medical professionals, wherein sharing permissions are enforced at the module level independently of insurer authorization requirements and within HIPAA-compliant access boundaries.

25. The system of claim 21, wherein the empathy measurement and support module generates, tracks, and acts upon Key Performance Indicators comprising at least empathy identification accuracy rate, emotional validation rate per interaction, staff resilience score over time, care continuity metric, and patient User Experience (UX) score, and wherein the module delivers real-time feedback and targeted intervention to the triage team based on KPI thresholds.

26. The system of claim 21, wherein the empathy measurement and support module assists the triage person in identifying and validating patient emotional states by prompting with structured feeling words drawn from the predefined emotional taxonomy, incorporating the RADAR listening formula with nonverbal warmth cues comprising physical positioning, postural openness, forward orientation, eye contact duration metric, and relaxation state indicator as additional inputs to the Empathy Score calculation when video interaction is active.

27. The system of claim 21, wherein the empathy measurement and support module incorporates trauma-informed design principles by providing built-in safeguards that protect emotionally vulnerable patients in pain from dominance, manipulation, or dismissal by either the human triage person or the AI system, while respecting the patient's voice, choice, and comfort level with AI involvement and maintaining licensed human oversight for final Advice, Refer, and Prescribe decisions.

28. The system of claim 21, wherein the AI-driven triage module is further configured to update its urgency scoring algorithm parameters based on anonymized, aggregated outcomes data from prior triage interactions across the physician's patient population, producing continuously improving triage accuracy without requiring manual rule updates.

29. The system of claim 21, wherein the billing and insurance processing module is further configured to flag CPT/ICD-10 code assignments that deviate from historical billing patterns for the same urgency score range and present the deviation to the physician for review prior to claim submission, thereby reducing billing errors and audit risk.

30. The system of claim 21, wherein the legal compliance and liability protection module is further configured to generate a timestamped, cryptographically signed interaction summary for each triage event comprising at least the urgency score, the Empathy Score, all escalation decisions, any vital signs captured, and the resulting CPT/ICD-10 code assignment, stored as an immutable record on the blockchain-backed ledger.

31. A method for continuous triage and medical coordination in a physician's practice, the method comprising: executing application instructions on a network-connected computing device comprising an AI-driven triage module, physician control module, billing module, EMR integration module, compliance module, ambient listening module, empathy measurement and support module, video escalation and real-time vital signs capture module, and patient data interoperability module; receiving a patient inquiry via at least one of a VoIP system, SMS gateway, mobile application, IoT devices, or web-based portal through a patient interface, wherein the method is audio-first such that initial contact is established via audio and safety assessment—including ambulance dispatch confirmation when clinically indicated—is completed prior to further escalation; classifying, via the ambient listening module during the live audio interaction, acoustic features of the patient comprising at least lung sounds, breathing rate, voice tensions, and coughing patterns against a predefined clinical acoustic taxonomy, identifying verbal disclosures not incorporated into the triage person's documented assessment, and generating real-time prompts delivered to the triage person; categorizing the inquiry via an urgency scoring algorithm configurable by the physician; routing to appropriate care or self-care advice based on physician-defined triage rules; escalating upon safety confirmation from audio to WebRTC video with dual-source vital signs capture, uploading all vital signs in real time to the patient's persistent data folder; measuring empathy levels of triage staff against a structured emotional taxonomy using the RADAR listening formula, generating quantified Empathy Scores, flagging degradation, and triggering supervisory interventions including wellness checks for volunteer and paid staff; applying AI-based medical coding to assign CPT and ICD-10 codes based on triage outcomes, cross-validating against payer-specific rules, and routing validated claims to the applicable payer channel with physician review and override; and recording the interaction via blockchain-backed immutable audit logs; wherein the method enables 24/7 patient access as a virtual extension of the physician's full state-licensed medical practice without geographic limitation, without insurer pre-approvals, and without disrupting the established physician-patient relationship.

32. A non-transitory computer-readable medium storing instructions that, when executed by a processor, cause a computing device to perform operations comprising: receiving a patient inquiry and routing it through an audio-first triage system that completes safety assessment—including ambulance dispatch confirmation when clinically indicated—prior to further escalation, using an AI-driven triage module configurable by the physician; classifying acoustic features of the patient and triage person captured during the live audio interaction against a predefined clinical acoustic taxonomy comprising lung sounds, breathing rate, voice tensions, and coughing patterns, identifying unaddressed verbal disclosures, and generating real-time prompts delivered to the triage person; determining an appropriate triage response via an urgency scoring algorithm; escalating from audio to WebRTC video with dual-source vital signs capture and real-time folder upload; measuring, scoring, and intervening on staff empathy levels using the RADAR listening formula and predefined emotional taxonomy, incorporating trauma-informed safeguards, generating quantified Empathy Scores, and tracking KPIs including empathy identification accuracy, emotional validation rates, staff resilience, care continuity, and patient UX metrics; applying AI-based medical coding to assign CPT and ICD-10 codes based on triage outcomes with physician review and override; recording interactions via AES-256 encryption and blockchain-backed immutable audit logs; and transmitting follow-up care instructions while maintaining the patient's perception of direct physician engagement; wherein the operations provide 24/7 triage as a virtual extension of the physician's state-licensed medical practice without geographic limitation.

33. The system of claim 21, wherein the billing and insurance processing module is further configured to: (i) derive CPT and ICD-10 code candidates directly from the numerical urgency rank and routing disposition generated by the urgency scoring algorithm upon triage completion;
(ii) automatically select from among the derived code candidates based on the patient's identified coverage type and applicable payer-specific coding rules; and (iii) present the selected CPT/ICD-10 code assignment to the physician via the secure GUI dashboard for review and override, and transmit the physician-approved code assignment to the applicable payer channel as a structured electronic claim, wherein the urgency scoring algorithm's numerical output serves as the primary clinical data input to the AI-based medical code selection process.

34. The system of claim 21, wherein the patient data interoperability module is further configured to, upon transfer of the patient interaction from a first triage staff member to a second staff member or physician, automatically transmit to the second staff member a consolidated interaction context record comprising: (i) the accumulated Empathy Score and flagged empathy threshold events from the current interaction; (ii) all verbal disclosures captured by the ambient listening module that were not incorporated into the first triage person's documented assessment; (iii) the current urgency score generated by the urgency scoring algorithm; and (iv) any acoustic features flagged as clinically significant during the interaction; and (v) any medication recommendations, prescription orders, or pharmacy routing decisions generated during the triage interaction—such that the second staff member receives complete and up-to-date interaction context, including the most recent clinical outputs, without requiring the patient to repeat prior disclosures.

35. The system of claim 21, wherein the patient interface is further configured to receive incoming communications from an authorized third-party caller acting on behalf of a patient who is unable to initiate contact independently—including credentialed community healthcare volunteers, family members, credentialed bystanders, or individuals acting under a valid Power of Attorney (limited or full)—authenticate the third-party caller's identity and legal authority who is unable to initiate contact independently, authenticate the third-party caller's identity and credential status, associate the third-party caller's reported observations with the patient's persistent data folder upon patient identity verification, and route the interaction through the same AI-driven triage module and ambient listening module as a direct patient-initiated contact, wherein the system records the third-party caller's identity and credential status as part of the immutable blockchain-backed audit trail for the interaction.

36. The system of claim 21, wherein the ambient listening module is configured to capture acoustic signals using a digital audio sampling rate sufficient to detect and analyze respiratory, cardiac, and vocal acoustic signals in the infrasound frequency range below 20 Hz—which is below the threshold of unaided human hearing—and wherein the module applies machine-learning-based acoustic analysis to the captured infrasound signals to identify clinically significant acoustic patterns including at least one of: (i) sub-audible lung sound abnormalities associated with pulmonary pathology; (ii) low-frequency cardiac sound components below the reliable detection threshold of the unaided human ear; or (iii) subaudible respiratory rhythm irregularities, and generates a clinical flag for each identified infrasound acoustic pattern and delivers the flag as a structured data output to the triage person and the patient's persistent data folder for physician review.

37. The system of claim 21, wherein the ambient listening module is further configured to capture sub-audible infrasound signals in the frequency range below 20 Hz through a barometric pressure sensor integrated in a consumer mobile device executing the patient interface, without requiring hardware modification or operating system kernel access, wherein the barometric pressure sensor detects respiratory pressure waves generated by the patient during the triage interaction, the module applies machine-learning-based pattern analysis to the captured pressure wave signals to identify respiratory rhythm irregularities and breathing rate anomalies clinically significant at the sub-audible frequency range, and delivers the identified anomalies as structured data outputs to the triage person and the patient's persistent data folder for physician review, wherein this infrasound capture pathway is deployable on commercially available mobile devices without requiring original equipment manufacturer (OEM) cooperation or hardware modification.
